(19) United States
(12) Patent Application Publication
(10) Pub. No.: US 2018/0116967 A1
(43) Pub. Date: May 3, 2018

(54) EXTENDED RELEASE TABLET OF CYCLOBENZAPRINE

(71) Applicant: SYNMOSA BIOPHARMA CORPORATION, Taibei (CN)

(72) Inventors: Pei-Chien SUN, Taibei (CN); Bor-Liang CHEN, Taibei (CN); Chia-Wen HUANG, Taibei (CN)

(21) Appl. No.: 15/860,330

(22) Filed: Jan. 2, 2018

Related U.S. Application Data
(63) Continuation-in-part of application No. PCT/CN2016/110395, filed on Dec. 16, 2016.

(30) Foreign Application Priority Data
Dec. 18, 2015 (CN) 201510961159.4

(51) Int. Cl. A61K 9/20; A61K 31/135; A61K 9/28; A61P 21/00; A61P 19/00

(52) U.S. Cl. CPC A61K 9/2095; A61K 31/135; A61K 9/2893; A61K 9/2018; A61P 19/00; A61K 9/2054; A61K 9/2013; A61P 21/00; A61K 9/2009

(57) ABSTRACT

A directly compressed extended release cyclobenzaprine tablet and method for making the tablet that includes homogenously mixing: (i) cyclobenzaprine, (ii) a filler selected from the group consisting of lactose, spray-dried lactose, mannitol, and combinations thereof; and (iii) a glidant selected from the group consisting of silica, peptized silica, and combinations thereof; and (iv) hydroxypropyl methylcellulose (HPMC) to provide a first mixture; homogenously mixing a lubricant with the first mixture to provide a second mixture; and directly compressing the second mixture into a tablet, having a ratio of filler to matrix forming polymer ranges from 1.66 to 2.07.

### CROSS-REFERENCE TO RELATED U.S. APPLICATIONS

This application is a continuation-in-part of International Application No. PCT/CN2016/110395, filed Dec. 12, 2016, now pending, the entire contents of which are expressly incorporated herein by reference.

### FIELD OF THE INVENTION

The present invention is directed to a cyclobenzaprine-containing extended-release pharmaceutical composition in a direct compression tablet, and methods of preparing the same.

### BACKGROUND OF THE INVENTION

Flexeril® is an immediate-release tablet containing cyclobenzaprine hydrochloride as the active agent, which has been approved by the U.S. Food and Drug Administration (FDA), that is used to cause a relaxing effect on skeletal muscles. The tablet is formed by mixing cyclobenzaprine hydrochloride with lactose, starch, magnesium stearate, and pigment, forming a tablet, and coating the tablet with a soluble coating. After oral administration of a 10-mg tablet, the average bioavailability of cyclobenzaprine is estimated to be 33% to 55%. About 93% of the drug is coupled to plasma protein.

The chemical name of cyclobenzaprine (which was first synthesized in 1961) is 3-(5H-dibenzo[a,d]cyclohepten-5-ylidene)-N,N-dimethyl-1-propanamine, and it has the structural formula depicted below as formula I.

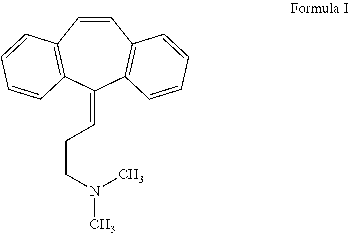 [SMILES: CN(C)CCC=C1c2ccccc2C=Cc2ccccc21]

It is well known that the drug is released quickly when an immediate-release cyclobenzaprine hydrochloride tablet is administered to patients with muscle spasms. Therefore, to maintain an effective pharmaceutical plasma concentration within the range needed to obtain the desired therapeutic effect, the immediate-release tablet is usually administered many times a day, which causes significant fluctuation in the drug's plasma concentration. For example, a 5 mg dose of the Flexeril® tablet (or increased to a 10 mg dose) administered 3 times/day is needed to show any relaxation effects.

According to clinical research, wherein a control group is referenced, and from information obtained from post marketing surveillance programs, it is known that the administration of cyclobenzaprine has serious adverse effects. According to the report of Borenstein D. G. et al. (Clinical Therapeutics, 2003, 25(4): 1056-1073), sedation side effects often occur when immediate-release cyclobenzaprine tablets are administered 3 times a day.

Extended-release dosage forms provide advantages compared to the immediate-release dosage forms. For example, extended-release dosage forms decrease variations in the plasma concentration-time profile, so as to reduce the side-effects often associated with immediate release dosage forms, thereby increasing patient compliance and therapeutic efficacy.

Cyclobenzaprine has a chemical structure that is similar to tricyclic antidepressants (TCA), which are known to exhibit adverse effects. Common adverse effects exhibited by cyclobenzaprine are, for example, drowsiness and thirst. For the elderly, myocardial infarction, glaucoma, arrhythmia, heart conduction interference, heart block, and heart failure can occur, and adverse events such as prolongation of the QT interval in the electrocardiogram and intraocular pressure can occur. Therefore, AMRIX®, an extended-release cyclobenzaprine formulation, formulated as an extended-release capsule filled with cyclobenzaprine-containing granules was marketed in 2007.

A. J. Weil et al. (Postgraduate Medical Journal, 2010 July; 122(4): 158-169) reported that most active agents show mild to moderate adverse reactions. The highest rate of adverse event occurrence for cyclobenzaprine occurs with the immediate release dosage form. Adverse event occurrence is 48.8% for the immediate-release dosage form, 39.7% for the 30-mg extended-release dosage form, 38.6% for the 15-mg extended-release dosage form, and 28.1% for placebo. The most common adverse event is thirst, where the incidence rate is 5.5% for the 15-mg extended-release dosage form, 13.5% for the 30-mg extended-release dosage form, 13.8% for the immediate-release dosage form, and 1.6% for placebo. Drowsiness is another common adverse event, wherein the incidence rate for patients with significant drowsiness is 7.3% for administration of the immediate-release tablet, 0.8% for the 15-mg extended-release dosage form, and 1.6% for the 30-mg extended-release dosage form.

U.S. Pat. Nos. 7,387,793 and 7,790,199 disclose multi-particulate beads that are an immediate-release particle core of cyclobenzaprine coated with a water insoluble polymer or a mixture of a water insoluble polymer and a small amount of a water soluble polymer. The water insoluble polymer can be cellulose acetate, polyvinyl acetate, Eudragit®, etc.

U.S. Patent Publication No. 2012/0064164 discloses an extended-release pharmaceutical composition that is a core coated with a layer of cyclobenzaprine that is then coated with one or more layers of an extended-release agent. Alternatively, cyclobenzaprine and the extended-release agents are mixed together and then coated onto the core to produce the extended-release particles. The water insoluble extended-release agent(s) provides the extended-release effect.

Korean Patent Publication No. 20120091748 discloses an extended-release pharmaceutical composition of cyclobenzaprine. The publication emphasizes that glyceryl behenate must be added when preparing the tablets, which also include ethyl cellulose and organic acids.

In view of the prior art, the inventors developed the present invention “Extended Release Tablet of Cyclobenzaprine.” The summary of the present invention is provided below.

### SUMMARY OF THE INVENTION

After studying commercial products and the literature, it can be seen that an extended-release dosage form of cyclobenzaprine is advantageous in that it can reduce the adverse effects that are commonly associated with immediate-release dosage forms of cyclobenzaprine. The present invention provides an extended-release tablet containing cyclobenzaprine that is easier and more cost effective to manufacture than prior art-known extended-release dosage forms. In particular, the tablet of the invention avoids the disadvantages associated with coated extended-release dosage forms, such as granule disintegration, coating inefficiency, and the need for solvent recycling.

One aspect of the present invention is an extended-release cyclobenzaprine-containing tablet that is made by direct compression. The term cyclobenzaprine, as used herein, means cyclobenzaprine or a pharmaceutically acceptable salt and/or solvate thereof. Preferably, the cyclobenzaprine is present as cyclobenzaprine hydrochloride. The tablet includes excipients such as an extended-release matrix forming material, a filler, a lubricant, and a glidant.

Another aspect of the present invention is an extended-release tablet composition made by direct compression that includes dantrolene sodium, methocarbamol, metaxalone, carisoprodol, diazepam or a pharmaceutically acceptable salt, solvate, and/or ester thereof. The tablet includes excipients, such as an extended-release matrix, a filler, a lubricant, and a glidant. The extended-release tablet is essentially the same as the cyclobenzaprine-containing tablet, but replaces the cyclobenzaprine with dantrolene sodium, methocarbamol, metaxalone, carisoprodol, diazepam, or a pharmaceutically acceptable salt, solvate and/or ester thereof.

The dissolution profile of the tablet is tested using United States Pharmacopeia (USP) dissolution apparatus I wherein the dissolution test is carried out in 900 mL of 0.1 N HCl at 37° C. and using a rotation speed of 50 rpm. The extended-release tablet exhibits a dissolution profile wherein about 30%-45% of active agent (preferably cyclobenzaprine) is released within 2 hours, about 45%-70% of active agent is released within 4 hours, and more than about 65% of the active agent is released within 8 hours.

In another aspect of the present invention, the extended-release tablet does not include an extended-release coating (film). It also is not necessary to include organic or inorganic acids or glyceryl behenate as a component of the extended-release tablet. In one embodiment, the extended-release tablet does not include organic or inorganic acids or glyceryl behenate.

Another aspect of the present invention is a method of preparing the cyclobenzaprine-containing extended-release tablet. The method involves: (i) homogenously mixing cyclobenzaprine or a pharmaceutically acceptable salt and/or solvate thereof with an extended-release matrix material, a filler, and a glidant to form a first mixture; (ii) homogenously mixing a lubricant with the first mixture to form a second mixture; and (iii) directly compressing the second mixture to form the extended-release tablet. It is not essential to add organic solvents, aqueous solvents, distilled water, or organic acids in any step of the process. The method is simple and cost effective.

The extended-release cyclobenzaprine tablet exhibits release of the cyclobenzaprine for between 8-12 hours when subjected to dissolution testing in 900 mL of 0.1 N HCl at 37° C. and a rotation speed of 50 rpm using USP dissolution test apparatus I.

### BRIEF DESCRIPTION OF THE DRAWINGS

FIG. 1 Depicts the dissolution profile for several cyclobenzaprine-containing dosage forms having different therapeutic utility. “A” illustrates the profile for an unsuitable dosage form, “B” illustrates the profile for a suitable dosage form, “C” illustrates the profile for a preferred dosage form, and “D” illustrates the profile for Amrix® ER.

FIG. 2 Depicts the dissolution profile for Tablets 06-09.

FIG. 3 Depicts the dissolution profile for Tablets 06-09 and 16.

FIG. 4 Depicts the dissolution profile for Tablets 10-13.

FIG. 5 Depicts the dissolution profile for Tablets 10-13 and 16.

FIG. 6 A diagram depicting the amounts of lactose and hydroxypropyl methylcellulose in tablets 13, 16, and 08.

FIG. 7 Depicts the dissolution profile for Tablets 08, 13, and 16.

FIG. 8 Depicts the dissolution profile for Tablets 12 and 27-29.

FIG. 9 Depicts the dissolution profile for Tablets 03 and 09.

FIG. 10 Depicts the dissolution profile for Tablets 12, 16, 18, and 21.

FIG. 11 Depicts the dissolution profile for Tablets Tablet 12 and the film-coated Tablets 14-15.

The dissolution profiles depicted in the figures were determined in 900 mL of 0.1 N HCl at 37° C. and a rotation speed of 50 rpm using USP dissolution test apparatus I.

### DETAILED DESCRIPTION OF THE PREFERRED EMBODIMENTS

Common methods for preparing tablets include wet granulation, dry granulation, and direct compression. In wet granulation, the active agent is combined and mixed with excipients (such as fillers, the glidants, and binders) in the presence of a solvent (typically water) to provide a mixture; the mixture is then dried and pulverized to provide granules; and the resulting granules, optionally combined with additional excipients, is compressed into a tablet. Because water or organic solvents are used during the preparation, heating or another drying method is needed. In dry granulation, the active agent is combined and mixed with excipients; the resulting mixture compacted and pulverized to provide granules, and the resulting granules, optionally combined with additional excipients, compressed into a tablet. Although water or organic solvents and the heating or drying step are excluded, compression and pulverization are still needed to form granules. In the direct compression process, the active agent is simply combined and mixed with various excipients and the resulting mixture directly compressed into a tablet.

The physical characteristics of a tablet, such as hardness, uniformity, disintegrative ability, and in vitro dissolution and, thus, the tablets efficacy as a therapeutic agent, are affected, in unpredictable ways, by the method of making the tablet and the selection of excipients.

The method of making the extended-release tablet of the present invention involves homogenously mixing the active agent with the excipients to provide a mixture and directly compressing the mixture into a tablet. The excipients may be sieved either individually or as a mixture before and/or after being combined with the active agent.

The method advantageously avoids mixing the active agent with solvents, in particular, organic solvents. When organic solvents are used, there may be residual organic solvent left in the final product. See Technical Guideline No. [H]GPH7-1 entitled “The technical guidelines for the studies on residual solvents in a chemical drug” published by the China Food and Drug Administration. The residual solvents can directly affect the quality and safety of the final product. Many organic solvents are harmful to the environment and to humans. In one aspect of the present invention the cyclobenzaprine-containing extended-release tablet is substantially free of organic solvents, and in another aspect is substantially free of solvents. The term “substantially free,” as used herein, means less than 10,000 ppm, preferably less than 1,000 ppm, and more preferably less than 100 ppm.

The filler used in the method of the invention can be lactose, spray-dried lactose, mannitol, or a combination thereof. Spray-dried lactose, which has good fluidity and compressibility and exhibits good stability high even if stored in humid conditions, is a preferred filler. The glidant can be silica, peptized silica, or a combination thereof. There are many pharmacologically acceptable silica products. Peptized silica (e.g., Aerosil® 200) is a preferred glidant. The lubricant can be magnesium stearate. Hydroxypropyl methylcellulose (HPMC) is a preferred extended-release matrix material and can be, for example, 90SH-4000SR (HPMC 4000) or 90SH-15000SR (HPMC 15000).

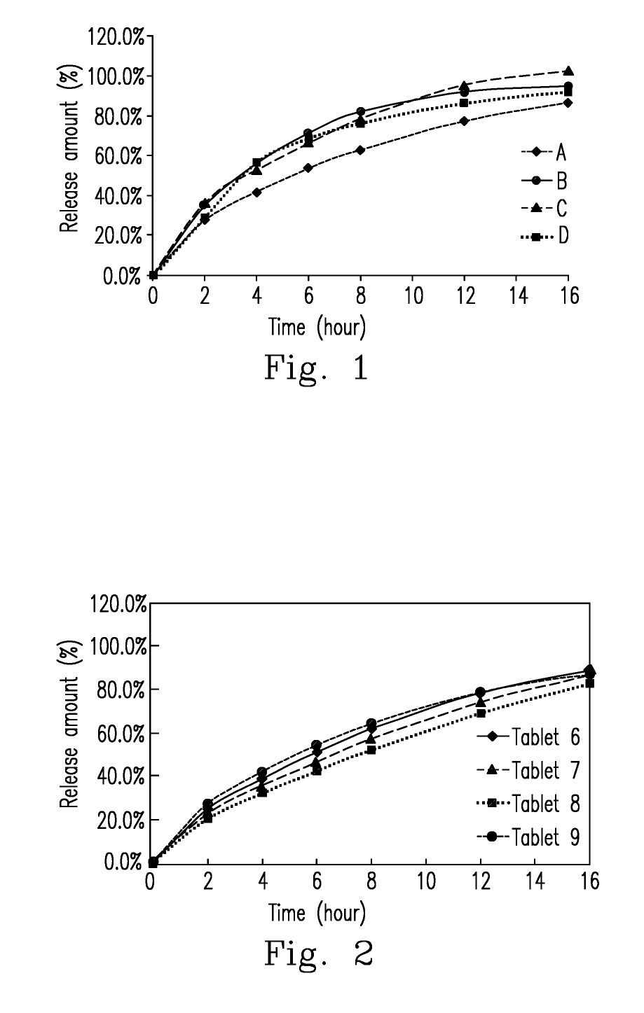

FIG. 1 illustrates the release profile for various cyclobenzaprine dosage forms determined in 900 mL of 0.1 N HCl at a temperature of 37° C. and a rotation speed of 50 rpm using USP apparatus (VK 7010 dissolution apparatus commercially available from Agilent Technologies). A illustrates the profile for an unsuitable dosage form, B illustrates the profile for a suitable dosage form, C illustrates the profile for a preferred dosage form, and D illustrates the profile for Amrix® ER.

In one embodiment, the release profile of the extended-release tablet of the invention does not deviate from the release profile exhibited by the commercially available extended-release cyclobenzaprine dosage form Amrix® ER by more than about 10% over 2 to 16 hours.

Table 1 summarizes the release profile exhibited by suitable and unsuitable dosage forms in 900 mL of 0.1 N HCl at a temperature of 37° C. and a rotation speed of 50 rpm using USP apparatus.

The dosage form releases about 30 to 45% over 2 hours, about 45 to 70% over 4 hours, and more than about 65% over 8 hours. Preferably, the dosage form releases about 30 to 45% over 2 hours, about 45% to 60% over 4 hours, and more than 70% over 8 hours. More preferably, the dosage form releases 32.2 to 36.6% over 2 hours, 51.0 to 58.2% over 4 hours, and 74.5 to 82.4% over 8 hours. If the amount released over 8-hours is less than 48.3%, the dosage form is determined as unacceptable.

TABLE 1
The released amount of the physiologically available dosage form in
clinical trials

| Type of the physiologically | Released amount | | |
|---|---|---|---|
| available dosage form | 2 Hours | 4 Hours | 8 Hours |
| Clinically suitable dosage | 30-45% | 45-70% | more than 65% |
| form | | | |
| Preferred dosage form | 30-45% | 45-60% | more than 70% |
| More preferred dosage form | 32.2-36.6% | 51.0-58.2% | 74.5-82.4% |
| Unsuitable dosage form | 26.0-27.4% | 35.9-41.6% | 48.3-62.9% |

A preferred composition of the cyclobenzaprine-containing extended-release tablet of in the present invention is provided in Table 2. The composition is prepared by accurately weighing the excipients lactose, silica, hydroxypropyl methylcellulose, and magnesium stearate; homogenously combining the excipients with the cyclobenzaprine to provide a mixture, and directly compressing the mixture to provide a tablet. Optionally, the excipients are sieved either individually and/or in combination and before or after being combined with the cyclobenzaprine. The tablet is formed by direct compression. The extended-release tablet can contain cyclobenzaprine in an amount of about 220-250 mg per tablet, wherein the amount of cyclobenzaprine is about 6% (w/w) to about 14% (w/w) of the total weight of the tablet; the amount of lactose is about 5% (w/w) to about 80% (w/w), preferably about 9% (w/w) to about 70% (w/w) of the total weight of the tablet, and more preferably about 35% (w/w) to about 65% (w/w) of the total weight of the tablet, e.g., 45% (w/w) to 65% (w/w) or 50% (w/w) to 65% (w/w) of the total weight of the tablet; the amount of hydroxypropyl methylcellulose is about 15% (w/w) to about 85% (w/w), preferably about 20% (w/w) to about 81% (w/w) of the total weight of the tablet, more preferably about 25% (w/w) to about 55% (w/w) of the total weight of the tablet, e.g., 20% (w/w) to 40% (w/w) or 25% (w/w) to 35% (w/w) of the total weight of the tablet; and the amount of silica (preferably peptized silica) is about 0.5% (w/w) to 2% (w/w) of the total weight of the tablet, preferably about 0.9% (w/w) of the total weight of the tablet; and the amount of lubricant (preferably magnesium stearate) is about 0.5% (w/w) to 3.5% (w/w), preferably about 1.3% (w/w) of the total weight of the tablet.

TABLE 2
An extended-release tablet of cyclobenzaprine

| Material | Amount (w/w) |
|---|---|
| Cyclobenzaprine hydrochloride | 6%-14% |
| Spray-dried lactose | 5%-80% |
| Hydroxypropyl methylcellulose | 15%-85% |
| 90SH-4000SR | |
| Peptized silica (Aerosil ® 200) | 0.5-2% |
| Magnesium stearate | 0.5%-3.5% |

The preferred method of making the extended-release tablet of the present invention involves (a) combining and homogenously mixing the cyclobenzaprine, spray-dried lactose, hydroxypropyl methylcellulose, and peptized silica to provide a first mixture, (b) combining and homogenously mixing magnesium stearate with the first mixture to provide a second mixture, and (c) directly compressing the second mixture into a tablet. The components of the tablet are accurately weighed before being combined. The components of the tablet may be sieved individually and/or in combination before or after being combined with the cyclobenzaprine.

The ratio of filler (preferably) lactose to extended-release matrix forming material (preferably hydroxypropyl methylcellulose) influences the release profile of the tablet. The formulae of Tablets 06-09 are shown in Table 3. The total amount of lactose and hydroxypropyl methylcellulose in each formula is about 90% of the total weight of the tablet. When the amount of hydroxypropyl methylcellulose in the total weight of the tablet is decreased from 81.81% to 50.00%, the amount of lactose in the total weight of the tablet is simultaneously increased from 9.09% to 40.91%. Table 4 and FIG. 2 depict the dissolution profile of Tablets 06-09.

TABLE 3
The formulae of Tablets 06-09

| Material (g) | Tablet 06 | Tablet 07 | Tablet 08 | Tablet 09 |
|---|---|---|---|---|
| Cyclobenzaprine | 15 | 15 | 15 | 15 |
| | (6.82%) | | | |
| Spray-dried lactose | 50 | 80 | 20 | 90 |
| | (22.73%) | (36.63%) | (9.09%) | (40.91%) |
| Hydroxypropyl | 150 | 120 | 180 | 110 |
| methylcellulose* | (68.18%) | (54.55%) | (81.81%) | (50.00%) |
| Peptized silica (Aerosil ® | 2 | 2 | 2 | 2 |
| 200) | (0.91%) | | | |
| Magnesium stearate | 3 | 3 | 3 | 3 |
| | (1.36%) | | | |
| mg/tablet | 220 | 220 | 220 | 220 |

*Hydroxypropyl methylcellulose is the type 90SH-4000SR

TABLE 4
The released amounts of Tablets 06-09

| Time | Released amount (%) | | | |
|---|---|---|---|---|
| (Hours) | Tablet 06 | Tablet 07 | Tablet 08 | Tablet 09 |
| 2 | 22.4 | 24.4 | 20.0 | 27.1 |
| 4 | 35.5 | 38.8 | 32.0 | 41.9 |
| 8 | 57.1 | 61.8 | 52.0 | 64.3 |
| 12 | 74.2 | 78.6 | 69.0 | 78.9 |
| 16 | 87.0 | 89.1 | 83.0 | 87.3 |

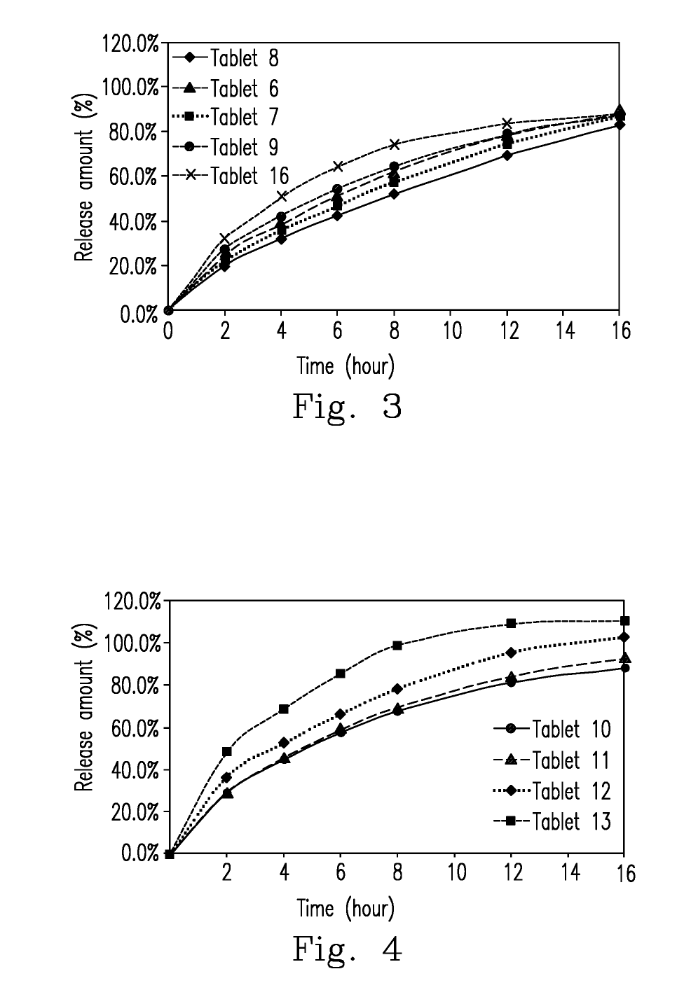

The amount of lactose in Tablets 08-09 is 9.09%-40.91% of the total weight of the tablet. The amount of lactose in Tablet 16 is 56.82% (shown in Table 5), but that of hydroxypropyl methylcellulose is less than any one of Tablets 06-09. FIG. 3 depicts the dissolution profile of Tablets 06-09 and Tablet 16.

Referring to Table 5, which shows the formulae of Tablets 10-13 and 16, the total amount of lactose and hydroxypropyl methylcellulose is 90.41% of the total weight of the tablet. Table 7 and FIG. 4 depict the dissolution profile of Tablets 10-13 as a function of time over 2-16 hours.

TABLE 5
The formulae of Tablets 10-13 and 16

| Material (g) | Tablet 10 | Tablet 11 | Tablet 12 | Tablet 13 | Tablet 16 |
|---|---|---|---|---|---|
| Cyclobenza- | 15 | 15 | 15 | 15 | 15 |
| prine | (6.82%) | | | | (6.82%) |
| Spray-dried | 100 | 110 | 135 | 155 | 125 |
| lactose | (45.45%) | (50.00%) | (61.36%) | (70.45%) | (56.82%) |
| Hydroxy- | 100 | 90 | 65 | 45 | 75 |
| propyl | (45.45%) | (40.91%) | (29.55%) | (20.45%) | (34.09%) |
| methyl- | | | | | |
| cellulose* | | | | | |
| Peptized | 2 | 2 | 2 | 2 | 2 |
| silica | (0.91%) | | | | (0.91%) |
| (Aerosil ® | | | | | |
| 200) | | | | | |
| Magnesium | 3 | 3 | 3 | 3 | 3 |
| stearate | (1.36%) | | | | (1.36%) |
| mg/tablet | 220 | 220 | 220 | 220 | 220 |

*Hydroxypropyl methylcellulose is the type 90SH-4000SR.

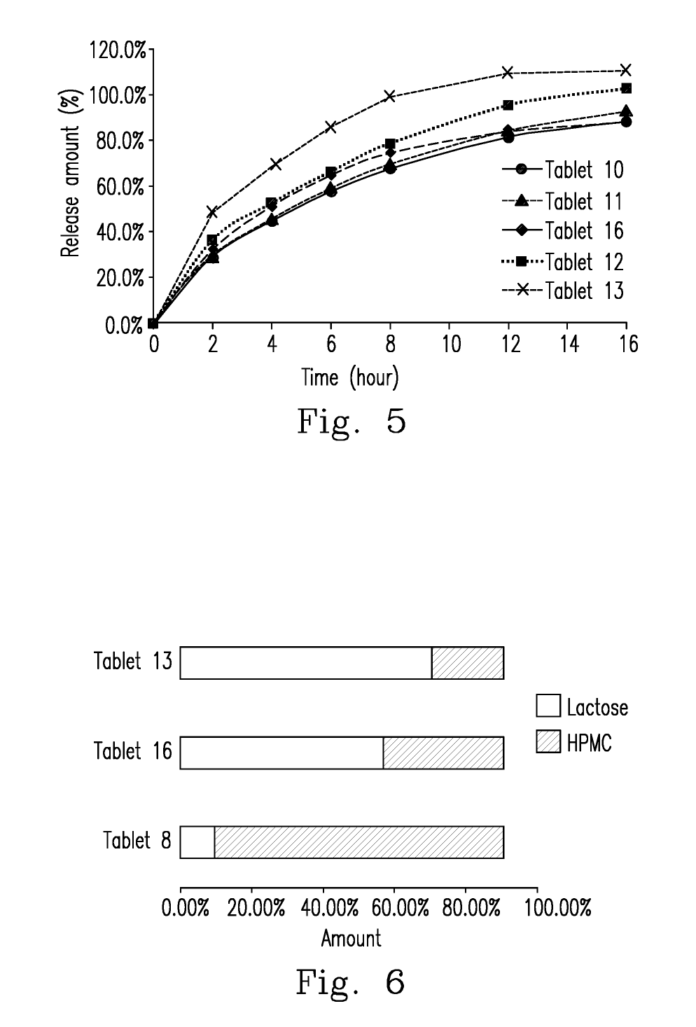

The amount of lactose in Tablet 10 is similar to the amount of hydroxypropyl methylcellulose. In Tablets 11, 16, 12, and 13 the amount of lactose is increased and that of hydroxypropyl methylcellulose is simultaneously decreased. As shown in Table 6, the ratio of HPMC:lactose is 33.33% in Tablet 06, 66.67% in Tablet 07, 11.11% in Tablet 08, 81.82% in Tablet 09, 122.22% in Tablet 11, 166.68% in Tablet 16, 207.69% in Tablet 12 and 344.44% in Tablet 13. Comparing the dissolution profile of Tablets 10-13 and 16 in FIG. 5 and Tablets 06-09 and 16 in FIG. 3, shows that only tablets 12 and 16 exhibit a dissolution profile that is considered suitable as defined in Table 1 (see also Table 7 and FIG. 5). Preferably, the extended-release composition has a ratio of extended-release matrix forming polymer:filler ranging from 1.66 to 2.07. In a more preferred embodiment, the extended-release matrix forming polymer is HPMC, the filler is lactose, and the ratio of HPMC:lactose ranges from 1.66 to 2.07.

TABLE 6
The ratios of two components of Tablets 06-09, 10-13 and 16

| Tablet | Ratio of two components* |
|---|---|
| 06 | 1:0.33 |
| 07 | 1:0.66 |
| 08 | 1:0.11 |
| 09 | 1:0.81 |
| 10 | 1:1 |
| 11 | 1:1.22 |
| 12 | 1:2.07 |
| 13 | 1:3.44 |
| 16 | 1:1.66 |

*The ratio of two components is the ratio of the amount of hydroxypropyl methylcellulose (HPMC) to that of spray-dried lactose, wherein the value for the amount of HPMC in the ratio is defined as 1.

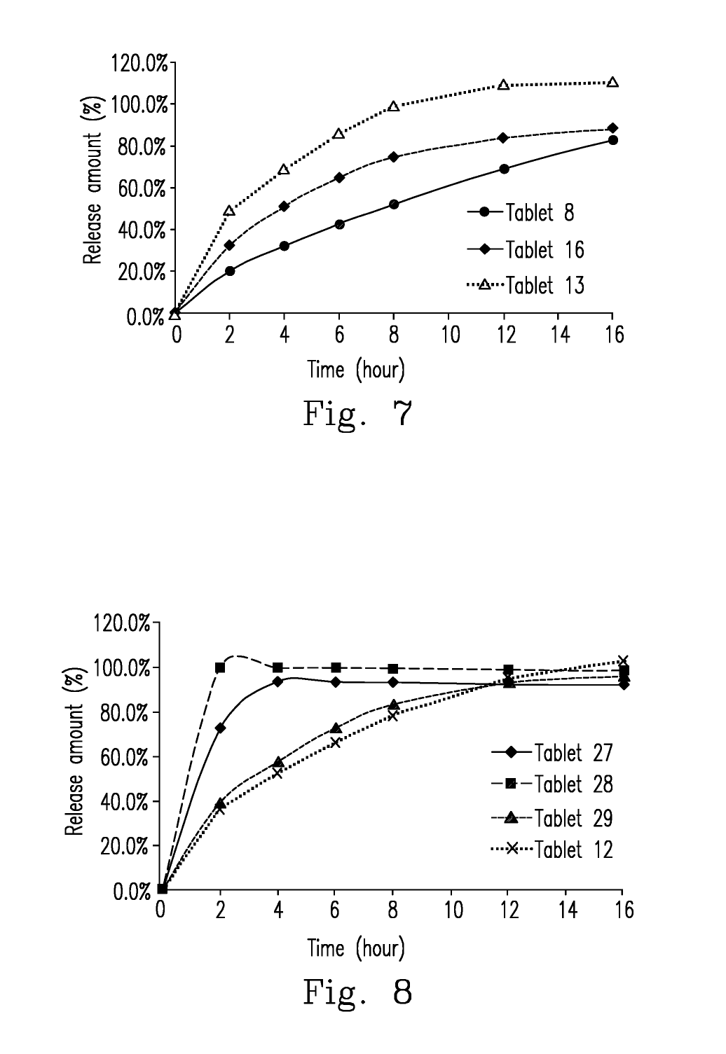

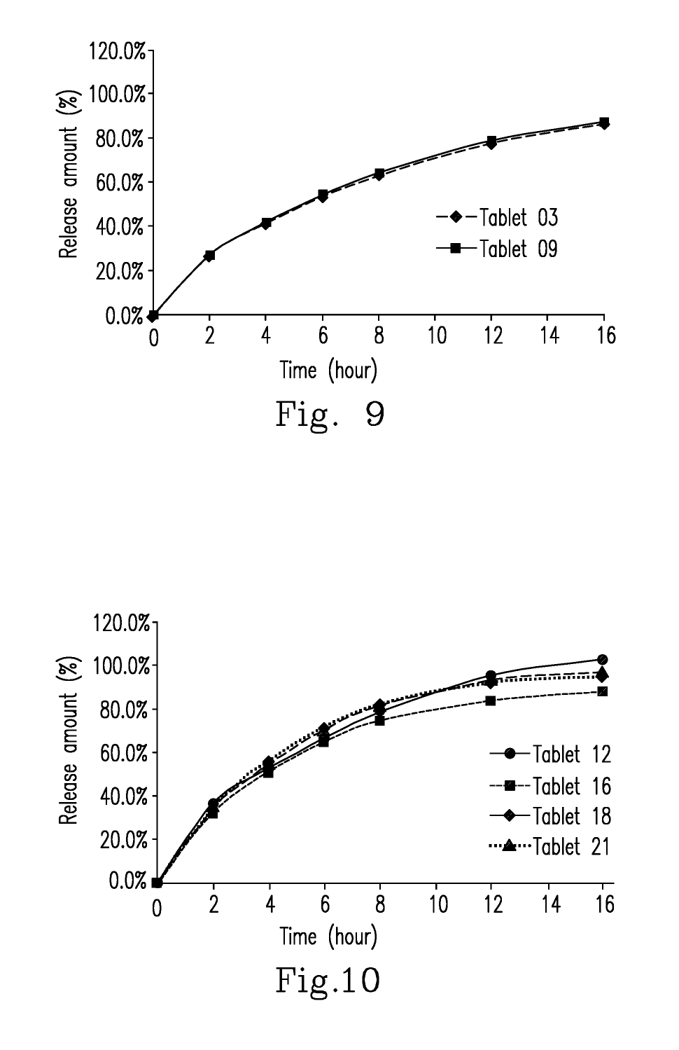

The total amount of lactose and hydroxypropyl methylcellulose in each of Tablets 8, 13, and 16 is about 90%. As depicted in FIG. 6, the amount of lactose is sequentially increased from Tablets 8, 16, and 13, and that of hydroxypropyl methylcellulose is correspondingly decreased. As shown in FIG. 7, comparing the dissolution profile of Tablets 8, 13, and 16, the weight ratio of lactose to hydroxypropyl methylcellulose affects how rapidly cyclobenzaprine is released from the tablets. Tablet 13 shows the fastest rate of release.

TABLE 7
The released amounts of Tablets 10-13 and 16

| Time | Release amount (%) | | | | |
|---|---|---|---|---|---|
| (Hours) | Tablet 10 | Tablet 11 | Tablet 12 | Tablet 13 | Tablet 16 |
| 2 | 29.0 | 29.3 | 36.5 | 48.5 | 32.2 |
| 4 | 44.7 | 45.6 | 52.6 | 68.6 | 51.0 |
| 8 | 67.5 | 69.4 | 78.4 | 98.9 | 74.5 |
| 12 | 81.3 | 84.3 | 95.4 | 109.2 | 83.7 |
| 16 | 88.2 | 92.8 | 102.8 | 110.4 | 88.1 |

The HPMC in Tablet 12 was replaced with different celluloses as the extended-release matrix material to provide Tablets 27-29 so as to evaluate how different celluloses affect the dissolution profile of the cyclobenzaprine. The formulae of Tablets 27-29 is provided in Table 8. As shown in Table 8, low-substituted hydroxypropyl cellulose (L-HPC) is used in Tablet 27, ethylcellulose is used in Tablet 28, and hydroxypropyl methylcellulose (HPMC) 15000 is used in Tablet 29. The components in each formula are directly compressed into tablets using the above described method.

As shown in Table 9 and FIG. 8, which depicts the dissolution profile of Tablets 27-29 from 2 hours to 16 hours, Tablet 29 has a dissolution profile similar to that of Tablet 12. The formula of Tablet 29, where hydroxypropyl methylcellulose (HPMC) 15000 is used as the extended-release matrix material, is similar to that of Tablet 12, where hydroxypropyl methylcellulose 90SH-4000SR is used as the extended-release matrix material. Hydroxypropyl methylcellulose is a preferred extended-release matrix material.

TABLE 8
The formulae of Tablets 27-29

| Material (g) | Tablet 27 | | Tablet 28 | Tablet 29 |
|---|---|---|---|---|
| Cyclobenzaprine | 15 | (6.82) | 15 | 15 |
| Spray-dried lactose | 135 | (61.36%) | 135 | 135 |
| The selected cellulose* | 65 | (29.55%) | 65 | 65 |
| Peptized silica | 2 | (0.91%) | 2 | 2 |
| (Aerosil ® 200) | | | | |
| Magnesium stearate | 3 | (1.36%) | 3 | 3 |

| | | | |
|---|---|---|---|
| mg/tablet | 220 | 220 | 220 |

*The selected cellulose is low-substituted hydroxypropyl cellulose in Tablet 27, ethylcellulose in Tablet 28 and HPMC 15000 in Tablet 29

TABLE 9
The released amounts of Tablets 27-29

| | Release amount (%) | | |
|---|---|---|---|
| Time (Hours) | Tablet 27 | Tablet 28 | Tablet 29 |
| 2 | 72.7 | 100.0 | 39.2 |
| 4 | 93.7 | 99.9 | 57.6 |
| 8 | 93.3 | 99.5 | 83.2 |
| 12 | 92.5 | 98.9 | 93.2 |
| 16 | 92.4 | 98.5 | 96.3 |

Table 10 shows the formulae of Tablets 01-03, which contain glyceryl behenate. Tablets 01-03 were found to be unsuitable as an extended-release tablet because they exhibited insufficient hardness and disintegrated too easily. Tablet 03 also exhibited capping.

TABLE 10
The formulae of Tablets 01-03

| Material (g) | Tablet 01 | Tablet 02 | Tablet 03 |
|---|---|---|---|
| Cyclobenzaprine | 15 | 15 | 15 |
| | (6.0%) | | (6.67%) |
| Synthesized aluminum silicate | 0 | 85 | 0 |
| Spray-dried lactose | 85 | 0 | 40 |
| | (34.0%) | | (17.78%) |
| Glyceryl behenate | 70 | 70 | 70 |
| | | (28.00%) | (31.11%) |
| Low-substituted hydroxypropyl | 70 | 70 | 0 |
| cellulose (L-HPC) | (28.00%) | (28.00%) | |
| Hydroxypropyl methylcellulose | 0 | 0 | 90 |
| 90SH-4000SR | | | (40.00%) |
| Peptized silica | 3 | 3 | 3 |
| (Aerosil ® 200) | (1.20%) | | (1.33%) |
| Magnesium stearate | 7 | 7 | 7 |
| | (2.80%) | | (3.11%) |
| mg/tablet | 250 | 250 | 225 |

Table 11 shows the formulae of Tablets 17, 18, and 21, which include citric acid. The combined amount of lactose, citric acid, and hydroxypropyl methylcellulose in Tablets 17, 18, and 21 are 91.48%, 90.92%, and 90.91% of the total weight of the tablet, respectively.

The amount of lactose and hydroxypropyl methylcellulose in Tablet 16 is 56.82% and 34.09% of the total weight of the tablet, respectively. The amount of lactose and hydroxypropyl methylcellulose in Tablet 12 is 61.36% and 29.55% of the total weight of the tablet, respectively. The amounts of lactose and hydroxypropyl methylcellulose in Tablet 18 is 56.82% and 34.09% of the total weight of the tablet, respectively. The amount of lactose and hydroxypropyl methylcellulose in Tablet 21 is 59.09% and 29.55% of the total weight of the tablet, respectively. Thus, the amount of lactose and hydroxypropyl methylcellulose in the citric acid-containing tablets of Examples 18 and 21 are similar to the amounts in citric acid-free tablets 16 and 12.

As shown in FIG. 10, Tablets 12, 16, 18, and 21 all exhibit a similar dissolution profile. However, when citric acid is included in the tablet, it results in an undesirable change in the appearance and color of the tablet. It is preferred that the tablet is free of organic and inorganic acids.

TABLE 11
The formulae of Tablets 17-18 and 21

| Material (g) | Tablet 17 | | Tablet 18 | | Tablet 21* | |
|---|---|---|---|---|---|---|
| Cyclobenzaprine | 15 | (6.38%) | 15 | | 15 | |
| Spray-dried lactose | 125 | (53.19%) | 125 | (56.82%) | 130 | (59.09%) |
| Hydroxypropyl | 75 | (31.91%) | 65 | (29.55%) | 65 | (29.55%) |
| methylcellulose | | | | | | |
| Citric acid | 15 | (6.38%) | 10 | (4.55%) | 5 | (2.27%) |
| Peptized silica | 2 | (0.85%) | 2 | | 2 | |
| (Aerosil ® 200) | | | | | | |

| | | | | |
|---|---|---|---|---|
| Magnesium stearate | 3 | (1.28%) | 3 | 3 |

| | | | |
|---|---|---|---|
| mg/tablet | 235 | 220 | 220 |

*Tablet 21 is prepared by mixing with 20 mL of 95% alcohol, followed by compression.

If desired, the tablets described above can further include a coating, such as a sugar coating or other non-functional film coating, to form a sugar coated tablet or film coated tablet. The coating can be applied using art-known methods for coating tablets. For example, a film coated Tablet 14 is formed by compressing the materials to form Tablet 12 and then coating the resulting tablet with film coating solution I. In addition, a film coated Tablet 15 is formed by compressing the materials to form Tablet 12 and then coating the resulting tablet with film coat solution II. The term “non-functional film coating” means a coating that does not cause extended-release of the active agent. Non-functional film coatings are typically used to simply improve the appearance of the tablet.

The difference between the film coat solution I and the film coat solution II is that hydroxypropyl methylcellulose (HPMC 606) is dissolved in alcohol in the former, and all components of the film coat are dissolved in the distilled water in the latter. In addition, the composition of the film coat solution I includes hydroxypropyl methylcellulose phthalate (HP-55), which is absent in the film coat solution II. Other differences between the compositions are shown in Table 12, wherein the amounts of titanium dioxide and yellow iron sesquioxide in the film coat solution II are slightly lower than the film coat solution I.

TABLE 12
The compositions of the film coat solutions

| Material (g) | Film coat solution I | Film coat solution II |
|---|---|---|
| HPMC 606 | 3.5 | 3.5 |
| PEG 6000 | 1.5 | 1.5 |
| HP-55 | 9 | 0 |
| Titanium dioxide | 0.77 | 0.38 |
| Iron sesquioxide | 0.2 | 0.1 |

The film coat solution I is prepared by combining 3.5 g hydroxypropyl methylcellulose (HPMC 606) dissolved in 104 mL of 95% alcohol, 1.5 g polyethylene glycol 6000 (PEG 6000) dissolved in 26 mL distilled water, 9 g hydroxypropyl methylcellulose phthalate (HPMCP, HP-55) dissolved in 50 mL distilled water to provide a solution, and adding 0.77 g titanium dioxide and 0.2 g yellow iron sesquioxide to the solution.

The film coat solution II is prepared by combining 3.5 g hydroxypropyl methylcellulose (HPMC 606) and 1.5 g polyethylene glycol 6000 (PEG 6000) in 50 mL distilled water, and adding 0.38 g titanium dioxide and 0.1 g yellow iron sesquioxide.

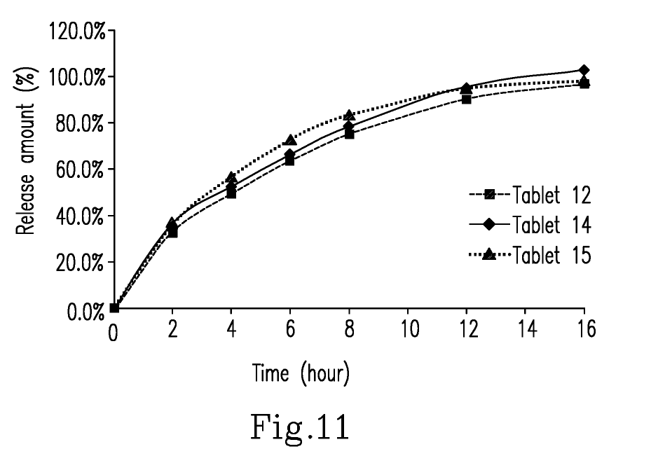

The film coated Tablet 14 is formed by compressing the materials to form tablet 12 followed by applying film coating solution I. The film coated Tablet 15 is formed by the same process except that film coating solution I is replaced by film coating solution II. As shown in Table 13 and FIG. 11, the difference between the dissolution profiles of Tablets 12, 14, and 15 in 900 mL of 0.1N HCl at 37° C. and a rotation speed of 50 rpm using USP apparatus is minimal.

TABLE 13
The release amounts of Tablets 12, 14 and 15

| | Release amount (%) | | |
|---|---|---|---|
| | | Film Coated | Film Coated |
| Time (Hours) | Tablet 12 | Tablet 14 | Tablet 15 |
| 2 | 36.5 | 32.9 | 34.2 |
| 4 | 52.6 | 49.3 | 56.8 |
| 8 | 78.4 | 74.90 | 76.0 |
| 12 | 95.4 | 90.2 | 85.0 |
| 16 | 102.8 | 96.6 | 88.1 |

The term “about” when associated with a range for a value means that the upper and lower limits of the range are approximate and are not limited to a expressly recited value. In other words, the range includes values slightly higher or lower than the expressly recited limits without departing from the scope of the present invention. The term “about,” as used in the specification, will be readily understood by a person of ordinary skill in the art. If the term “about” would not be readily understood by a person of ordinary skill in the art, the term “about” may be taken to mean the expressly recited value±10%.

The terms “drug”, “active component”, “active agent”, “main drug component”, “main active component” and the like mean a component, such as a compound, an antibody, a protein, or the like, that has an effect on physiological activity. These terms are interchangeable, and their meanings are the same.

The terms “extended-release”, “slow release” and so on have their art-recognized meaning, are interchangeable, and have the same meaning. The terms “free of solvent or solvent mixture” and “free of solvent” mean that the process for preparing a tablet does not use organic solvents, aqueous solvents, or solvent mixtures.

The terms “excipient” or “pharmaceutically acceptable carrier or excipient” and “bioavailable carrier or excipient” mean any components, other than the active agent, included in the tablets. These components include, for example, known excipients such as, dispersants, coatings, preservatives, and delayed absorbents. In general, the carrier or the excipient themselves do not exhibit any therapeutic efficacy. Preferably, the pharmaceutically acceptable carrier or excipient when administered to an animal or human does not result in a reaction, allergic response, or other undesirable effect.

In summary, all technical and scientific terms in the specification have their common meaning as would be readily understood by a person of ordinary skill in the art unless the term is otherwise defined. The present invention will now be described more specifically with reference to the following Examples, which are illustrative and not intended to be limiting. The active agents and materials used in the present invention are commercially available.

The present invention is an extended-release cyclobenzaprine-containing tablet, wherein cyclobenzaprine or a pharmaceutically acceptable salt and/or solvate, thereof is combined with an extended-release matrix material, a filler, a glidant and a lubricant. The tablet is prepared by homogenously mixing the components, without adding organic solvents, aqueous solvents, distilled water, or organic acids, to provide a mixture, and directly compressing the resulting mixture into the extended-release tablet.

The present invention can be realized by the following Examples, and can be accomplished by a person of ordinary skill in the art according to the disclosure. The following Examples, however, are illustrative and are not intended to be limiting.

### EXAMPLES

### Example 1

### Extended-Release Tablet of Cyclobenzaprine

| Material | Actual dosage (mg) |
|---|---|
| Cyclobenzaprine | 15 |
| Spray-dried lactose | 135 |
| Hydroxypropyl methylcellulose | 65 |
| 90SH-4000SR | |
| Peptized silica (Aerosil ® 200) | 2 |
| Magnesium stearate | 3 |

An extended-release tablet is prepared by (a) accurately weighing cyclobenzaprine, lactose, hydroxypropyl methylcellulose 90SH-4000SR, and peptized silica, and homogenously mixing and sieving the components to provide a first mixture; (b) sieving magnesium stearate and homogenously mixing the magnesium stearate with the first mixture to provide a second mixture, and then sieving the second mixture; and (c) directly compressing the second mixture into tablets. The method does not use any organic solvents, aqueous solvents, or distilled water. The dissolution rate of the resulting tablet in 900 mL of 0.1N HCl at 37° C. and a rotation speed of 50 rpm using USP apparatus I is similar to that of the commercial product (Amrix® ER capsules).

### Example 2

### The Preparation of Tablets 06-09

The formulae of Tablets 06-09

| Material (g) | Tablet 06 | Tablet 07 | Tablet 08 | Tablet 09 |
|---|---|---|---|---|
| Cyclobenzaprine | 15 | 15 | 15 | 15 |
| Spray-dried lactose | 50 | 80 | 20 | 90 |
| Hydroxypropyl | 150 | 120 | 180 | 110 |
| methylcellulose | | | | |
| Peptized silica | 2 | 2 | 2 | 2 |
| (Aerosil ® 200) | | | | |
| Magnesium stearate | 3 | 3 | 3 | 3 |
| mg/tablet | 220 | 220 | 220 | 220 |

The tablets of Examples 2 (tablets 06-09) are prepared using the method described in Example 1.

### Example 3

### The Preparation of Tablets 10-13

The formulae of Tablets 10-13

| Material (g) | Tablet 10 | Tablet 11 | Tablet 12 | Tablet 13 |
|---|---|---|---|---|
| Cyclobenzaprine | 15 | 15 | 15 | 15 |
| Spray-dried lactose | 100 | 110 | 135 | 155 |
| Cellulose* | 100 | 90 | 65 | 45 |
| Silica* | 2 | 2 | 2 | 2 |
| Magnesium stearate | 3 | 3 | 3 | 3 |

*The cellulose used in Tablets 10-13 is hydropropyl methylcellulose 90SH-4000SR, and the silica used in Tablets 10-13 is peptized silica (Aerosil ® 200).

The tablets of Examples 3 (tablets 10-13) are prepared using the method described in Example 1.

### Example 4

### The Preparation of Tablets 16 and 29

The formulae of Tablets 16 and 29

| | Actual dosage (g) | |
|---|---|---|
| Material | Tablet 16 | Tablet 29 |
| Cyclobenzaprine | 15 | 15 |
| Spray-dried lactose | 125 | 135 |
| Hydroxypropyl methylcellulose* | 75 | 65 |
| Peptized silica (Aerosil ® 200) | 2 | 2 |
| Magnesium stearate | 3 | 3 |
| mg/tablet | 220 | 220 |

*The hydroxypropyl methylcellulose used in Tablet 16 and 29 is 90SH-4000SR and HPMC 15000, respectively.

The tablets of Example 4 (tablets 16 and 19) are prepared using the method described in Example 1.

### Example 5

### Example of an Unsuitable Dosage Form

| Material | Amount per unit (mg/tablet) |
|---|---|
| Cyclobenzaprine | 15 |
| Lactose | 125 |
| Citric acid | 10 |
| Hydroxypropyl methylcellulose | 65 |
| 90SH-4000SR | |
| Peptized silica (Aerosil ® 200) | 2 |
| Magnesium stearate | 3 |

An extended-release tablet including components in the amounts listed in the table above is prepared by (a) homogenously mixing cyclobenzaprine, lactose, citric acid, hydroxypropyl methylcellulose 90SH-4000SR and peptized silica and sieving the components to provide a first mixture; (b) sieving magnesium stearate and homogenously mixing the magnesium stearate with the first mixture to provide a second mixture, and then sieving the second mixture; and (c) directly compressing the second mixture into tablets.

### Example 6

### Unsuitable Dosage Form

| Material | Amount per unit (mg/tablet) |
|---|---|
| Cyclobenzaprine | 15 |
| Lactose | 40 |
| Glyceryl behenate | 70 |
| Hydroxypropyl methylcellulose | 90 |
| 90SH-4000SR | |
| Peptized silica (Aerosil ® 200) | 3 |
| Magnesium stearate | 7 |

An extended-release tablet having components in the amounts listed in the table above is prepared by (a) homogenously mixing cyclobenzaprine, lactose, glyceryl behenate, hydroxypropyl methylcellulose 90SH-4000SR and peptized silica and sieving the components to provide a first mixture; (b) sieving magnesium stearate and homogenously mixing the magnesium stearate with the first mixture to provide a second mixture and then sieving the second mixture; and (c) directly compressing the second mixture into tablets. It is found that the hardness of the resulting tablet formed by direct compression is unsatisfactory, and thus the tablet is unsuitable.

What is claimed is:

1. A method for preparing an extended-release tablet, comprising:
a. homogenously mixing:
(i) cyclobenzaprine,
(ii) a filler selected from the group consisting of lactose, spray-dried lactose, mannitol, and combinations thereof; and
(iii) a glidant selected from the group consisting of silica, peptized silica, and combinations thereof; and
(iv) hydroxypropyl methylcellulose (HPMC),
to provide a first mixture;
b. homogenously mixing a lubricant with the first mixture to provide a second mixture; and
c. directly compressing the second mixture into a tablet;
wherein the amount of cyclobenzaprine is from about 6% (w/w) to about 14% (w/w) of the total weight of the tablet, the amount of filler is from about 5% (w/w) to about 80% (w/w) of the total weight of the tablet, the amount of hydroxypropyl methylcellulose is from about 15% (w/w) to about 85% (w/w) of the total weight of the tablet, the amount of glidant is from about 0.5% (w/w) to about 2% (w/w) of the total weight of the tablet, and the amount of lubricant is from about 0.5% (w/w) to 3.5% (w/w) of the total weight of the tablet; and
wherein the ratio of filler to matrix forming polymer ranges from 1.66 to 2.07.

2. The method of claim 1, wherein the amount of filler is from about 9% (w/w) to about 70% (w/w) of the total weight of the tablet, the amount of hydroxypropyl methylcellulose is from about 20% (w/w) to about 81% (w/w) of the total weight of the tablet, the amount of glidant is about 0.9% (w/w) of the total weight of the tablet, and the amount of lubricant is about 1.3% (w/w) of the total weight of the tablet.

3. The method of claim 2, wherein the filler comprises lactose or spray-dried lactose, or a combination thereof, present in an amount from about 35% (w/w) to about 65% (w/w) of the total weight of the tablet, and the amount of hydroxypropyl methylcellulose is from about 25% (w/w) to about 55% (w/w) of the total weight of the tablet.

4. The method of claim 1, wherein the percentage of cyclobenzaprine released from the tablet at 2 hours ranges from 30% to 45%, the percentage of cyclobenzaprine released from the tablet at 4 hours ranges from 45% to 70%, and the percentage of cyclobenzaprine released from the tablet at 8 hours is more than 65% when the tablet is subject to dissolution testing in 900 mL of 0.1N HCl at 37° C. and a rotation speed of 50 rpm using USP apparatus I.

5. The method of claim 4, wherein the percentage of cyclobenzaprine released from the tablet at 2 hours ranges from 32.2% to 36.6%, the percentage of cyclobenzaprine released from the tablet at 4 hours ranges from 51 to 58.2%, and the percentage of cyclobenzaprine released from the tablet at 8 hours ranges from 74.5 to 82.4%.

6. The method of claim 1, wherein the lubricant is magnesium stearate.

7. The method of claim 6, wherein the filler is spray-dried lactose and the glidant is peptized silica.

8. The method of claim 1, wherein the filler is spray-dried lactose and the glidant is peptized silica.

9. The method of claim 1, wherein the amount of cyclobenzaprine is about 6.82% (w/w) of the total weight of the tablet, the filler is spray-dried lactose in an amount ranging from about 56.82% (w/w) to about 61.36% (w/w) of the total weight of the tablet, the amount of hydroxypropyl methylcellulose ranges from about 29.55% (w/w) to about 34.09% (w/w) of the total weight of the tablet, the glidant is peptized silica in an amount of about 0.91% (w/w) of the total weight of the tablet, and the lubricant is magnesium stearate in an amount of about 1.36% (w/w) of the total weight of the tablet.

10. The method of claim 1, wherein the tablet does not include an inorganic or organic acid.

11. The method of claim 1, further comprising disposing a sugar coating or a non-functional coating over the tablet.

12. An extended-release tablet prepared by the method of claim 1.

13. A method of causing muscle relaxation in a human, which comprises administering to the human the extended-release tablet of claim 12.

14. A method of treating a skeletal muscle disease or condition in a human, which comprises administering to the human the extended-release tablet of claim 12.

15. An extended-release tablet, comprising:
a. cyclobenzaprine;
b. a filler selected from the group consisting of lactose, spray-dried lactose, mannitol, and combinations thereof;
c. a glidant selected from the group consisting of silica, peptized silica, and combinations thereof;
d. hydroxypropyl methylcellulose (HPMC); and
e. a lubricant
wherein the tablet is substantially free of organic solvent;
wherein the amount of cyclobenzaprine ranges from about 6% (w/w) to about 14% (w/w) of the total weight of the tablet, the amount of lactose ranges from about 9% (w/w) to about 70% (w/w) of the total weight of the tablet, the amount of hydroxypropyl methylcellulose ranges from about 20% (w/w) to about 81% (w/w) of the total weight of the tablet, the amount of glidant ranges from about 0.5% (w/w) to about 2% (w/w) of the total weight of the tablet, and the amount of lubricant ranges from about 0.5% (w/w) to 3.5% (w/w) of the total weight of the tablet; and
wherein the ratio of filler to hydroxypropyl methylcellulose ranges from 1.66 to 2.07.

16. The tablet of claim 15, wherein the lactose is present in an amount from about 35% (w/w) to about 65% (w/w) of the total weight of the tablet, the hydroxypropyl methylcellulose is present in an amount from about 25% (w/w) to about 55% (w/w) of the total weight of the tablet, the glidant is present in an amount of about 0.9% (w/w) of the total weight of the tablet, and the lubricant is present in an amount of about 1.3% (w/w) of the total weight of the tablet.

17. The tablet of claim 15, wherein the tablet does not include an inorganic or organic acid.

18. The tablet of claim 15, wherein the amount of cyclobenzaprine released from the tablet at 2 hours ranges from 30% to 45%, the amount of cyclobenzaprine released from the tablet at 4 hours ranges from 45% to 60%, and the amount of cyclobenzaprine released from the tablet at 8 hours is more than 70% when the tablet is subject to dissolution testing in 900 mL of 0.1N HCl at 37° C. and a rotation speed of 50 rpm using USP apparatus I.

19. The tablet of claim 18, wherein the amount of cyclobenzaprine released from the tablet at 2 hours ranges from 32.2% to 36.6%, the amount of cyclobenzaprine released from the tablet at 4 hours ranges from 51.0 to 58.2%, and the amount of cyclobenzaprine released from the tablet at 8 hours ranges from 74.5 to 82.4%.

20. The tablet of claim 15, wherein the filler is spray-dried lactose, the glidant is peptized silica, and the lubricant is magnesium stearate.

21. The tablet of claim 15, wherein the amount of cyclobenzaprine is about 6.82% (w/w) of the total weight of the tablet, the filler is spray-dried lactose in an amount ranging from about 56.82% (w/w) to about 61.36% (w/w) of the total weight of the tablet, the amount of hydroxypropyl methylcellulose ranges from about 29.55% (w/w) to about 34.09% (w/w) of the total weight of the tablet, the glidant is peptized silica in an amount of about 0.91% (w/w) of the total weight of the tablet, and the lubricant is magnesium stearate in an amount of about 1.36% (w/w) of the total weight of the tablet.

22. The tablet of claim 15, further comprising disposing a sugar coating or a non-functional coating over the tablet.

23. A method of causing muscle relaxation in a human, which comprises administering to the human the extended-release tablet of claim 15.

24. A method of treating a skeletal muscle disease or condition in a human, which comprises administering to the human the extended-release tablet of claim 15.
